(19) United States
(12) Patent Application Publication
(10) Pub. No.: US 2021/0322484 A1
(43) Pub. Date: Oct. 21, 2021

(54) MACROMOLECULAR CLUSTERS OF CARDIAC STEM CELLS AND METHODS FOR MAKING AND USING THEM

(71) Applicant: SAN DIEGO STATE UNIVERSITY (SDSU) FOUNDATION, DBA SAN DIEGO STATE UNIVERSITY RESE, San Diego, CA (US)

(72) Inventors: Mark A. SUSSMAN, San Diego, CA (US); Megan M. MONSANTO, San Diego, CA (US)

(21) Appl. No.: 17/348,038

(22) Filed: Jun. 15, 2021

Related U.S. Application Data
(62) Division of application No. 14/940,071, filed on Nov. 12, 2015.
(60) Provisional application No. 62/080,003, filed on Nov. 14, 2014.

(51) Int. Cl. A61K 35/34; A61K 35/28; A61K 35/44; C12N 5/077

(52) U.S. Cl. CPC A61K 35/34; A61K 35/28; A61K 35/44; C12N 2513/00; C12N 2502/28; C12N 2502/1352; C12N 5/0657

(57) ABSTRACT

In alternative embodiments, provided are macrocellular structures or artificially configured plurality of cells, the so-called “cardioclusters” as provided herein, comprising: a core region or cluster: comprising a plurality of first cardiac stem cells or cardiac progenitor cells; and a second region or a peripheral region positioned at least partially surrounding the outer surface of the core region or cluster, or at least partially around the core region or cluster, comprising a plurality of second cardiac stem cells; and methods for making and using them. In alternative embodiments, the second cardiac progenitor cells are cardiac progenitor cells or cardiac stem cells, mesenchymal stem cells or mesenchymal progenitor cells, or endothelial progenitor cells or endothelial stem cells.

### RELATED APPLICATIONS

This U.S. utility patent application claims benefit of priority under 35 U.S.C. § 119(e) of U.S. provisional patent application Ser. No. (USSN) 62/080,003, filed Nov. 14, 2014. The aforementioned application is expressly incorporated herein by reference in its entirety and for all purposes.

### STATEMENT AS TO FEDERALLY SPONSORED RESEARCH

This invention was made with government support under grant numbers NIH 1R01HL122525-01, National Institutes of Health (NIH), DHHS. The government has certain rights in the invention.

### TECHNICAL FIELD

This invention generally relates to cell and molecular and stem cell biology and regenerative medicine. In alternative embodiments, provided are macrocellular structures or artificially configured organized groupings of a plurality of cells, the so-called “cardioclusters” as provided herein, comprising: a core region or cluster: comprising a plurality of first cardiac stem cells or cardiac progenitor cells; and a second region or a peripheral region positioned at least partially surrounding the outer surface of the core region or cluster, or at least partially around the core region or cluster, comprising a plurality of second cardiac stem cells; and methods for making and using them. In alternative embodiments, the second cardiac progenitor cells are cardiac progenitor cells or cardiac stem cells, mesenchymal stem cells or mesenchymal progenitor cells, or endothelial progenitor cells or endothelial stem cells. In alternative embodiments, the macrocellular structures or artificially configured plurality of cells further comprise a plurality of third stem cells or progenitor cells that are different from the first cardiac stem cells or progenitor cells and the second cardiac stem cells or progenitor cells. These third stem cells can be cardiac progenitor cells or cardiac stem cells, mesenchymal stem cells or mesenchymal progenitor cells, or endothelial progenitor cells or endothelial stem cells.

In alternative embodiments, provided are products of manufacture comprising one or more of the macrocellular structures or an artificially configured plurality of cells, the so-called “cardioclusters” as provided herein, wherein optionally the product of manufacture further comprises a drug delivery device, an implant, a catheter, a stent, or a medical device.

In alternative embodiments, provided are methods for inducing cardiogenesis in the mammalian heart, or for treating or ameliorating a heart genetic defect, injury or dysfunction, or an injury or dysfunction subsequent to an ischemic injury or a heart failure, or an injury or dysfunction resulting from a myocardial infarction (MI), comprising administering to an individual in need thereof one or more of the macrocellular structures or an artificially configured plurality of cells, the so-called “cardioclusters” provided herein, or a product of manufacture provided herein.

### BACKGROUND

Cellular therapy using stem cells derived from the bone marrow and cells of cardiac origin are validated to treat damage after myocardial infarction (MI) in both small animal models and human clinical trials. Application of cellular therapy of MI is hindered by results of little to no improvement in cardiac function after long-term follow up studies using a variety of stem cell strategies. The inherent limitation of autologous stem cell therapy is that cells derived from aged organs have increased expression of senescent markers and acquisition of chromosomal abnormalities leading to undesirable cellular characteristics such as slowed proliferation and increased susceptibility to cellular death. Furthermore, based on animal models, cellular survival and engraftment is hindered by adverse inflammation, inhibiting the ability of transplanted stem cells to efficiently differentiate into cardiac cells. Improvement of stem cell engraftment and survival has been attempted by co-injection of stem cells with biomaterials, cytokines and growth factors, or genetically enhancing cells with pro-survival and anti-apoptotic genes.

The heart is capable of limited regeneration, as evidenced by cardiomyocyte re-entry into the cell cycle and production of new mono-nucleated myocytes during aging and after pathological damage. New myocyte formation is partially due to reserve c-kit⁺ cardiac progenitor cells (CPCs) found in complex microenvironments or niches. In vivo, CPCs retain expression of primitive cardiac transcription factors and upon activation can give rise to cells of the cardiac lineages.

The regenerative potential of stem cells in a clinical setting is still largely unrealized, as stem cells are suggested to function through a variety of mechanisms for myocardial repair yet stem cells are inherently limited because of origin and potency status. Although approaches using genetic manipulations and micropatterning of cellular structures have been evaluated, the central point to improve regeneration has been lost with focus on a favorite stem cell rather than the optimal stem cell population(s).

### SUMMARY

In alternative embodiments, provided are a macrocellular structure, a cardiocluster of cells, or an artificially configured plurality of cells, comprising:
a core region or cluster: comprising a plurality of first cardiac stem cells or cardiac progenitor cells; and
a second region or a peripheral region positioned: at least partially surrounding the outer surface of the core region or cluster, or at least partially around the core region or cluster, comprising a plurality of second cardiac stem cells, wherein the first cardiac stem cells and the second cardiac stem cells are different, and the second cardiac progenitor cells are selected from the group consisting of:
a plurality of cardiac progenitor cells or cardiac stem cells,
a plurality of mesenchymal stem cells or mesenchymal progenitor cells,
a plurality of endothelial progenitor cells or endothelial stem cells, and
a combination thereof.

In alternative embodiments the macrocellular structures, cardioclusters of cells, or artificially configured plurality of cells further comprise a plurality of third stem cells or progenitor cells that are different from the first cardiac stem cells or progenitor cells and the second cardiac stem cells or progenitor cells, and optionally the plurality of third stem cells are selected from the group consisting of:
a plurality of cardiac progenitor cells or cardiac stem cells,
a plurality of mesenchymal stem cells or mesenchymal progenitor cells,
a plurality of endothelial progenitor cells or endothelial stem cells, and
a combination thereof,

and optionally the plurality of third stem cell are positioned or configured: at least partially surrounding the outer surface of the second region or the core region or cluster, or at least partially around the second region or the core region or cluster,

and optionally the plurality of third stem cells or progenitor cells are positioned or configured in the core region or cluster,

and optionally the plurality of third stem cell are positioned or configured: at least partially surrounding the outer surface of the second region or peripheral region or the core region or cluster, or at least partially around the second region or peripheral region or the core region or cluster, and are also positioned or configured in the core region or cluster,

and optionally the plurality of third stem cells or progenitor cells are of non-cardiac origin, and optionally the third stem cells are mesenchymal stem cells of non-cardiac origin.

In alternative embodiments, the first, second, and/or third stem cells are of human origin, mammalian origin, or of non-human origin.

In alternative embodiments, the third stem cells are mesenchymal stem cells of cardiac origin. In alternative embodiments, the first and second cardiac stem cells are of cardiac origin. In alternative embodiments, the plurality of third stem cells are mesenchymal stem cells of cardiac origin.

In alternative embodiments, the core region or cluster comprises a plurality of mesenchymal stem cells and the second region or peripheral region comprises a plurality of cardiac progenitor cells.

In alternative embodiments, the core region or cluster comprises a plurality of mesenchymal stem cells and the second region or peripheral region comprises a plurality of endothelial progenitor cells, or, the core region or cluster further comprises cardiac progenitor cells.

In alternative embodiments, the core region or cluster, and/or the second region or peripheral region, comprises cells selected from the group consisting of: c-kit+ cardiac progenitor cells (CPCs), CD90+/CD105+ mesenchymal stem cells (MSCs) CD133+ endothelial progenitor cells (EPCs), and a combination thereof.

In alternative embodiments, provided are methods for making: a cardiocluster of cells, a macrocellular structure or an artificially configured plurality of cells, a macrocellular structure, a cardiocluster of cells or an artificially configured plurality of cells as provided herein, comprising:

providing a plurality of first cardiac stem cells, and forming or fabricating a core region or cluster of cells comprising the plurality of first cardiac stem cells; and

providing a plurality of second cardiac stem cells, and forming or fabricating a second region or a peripheral region comprising: a layer comprising a plurality of the second cardiac stem cells at least partially around the core region or cluster of cells, or placing the second cardiac stem cells at least partially around the outer surface of the region or cluster of cells,

wherein the first cardiac stem cells and the second cardiac stem cells are different, and the second cardiac stem cells are selected from the group consisting of: a plurality of cardiac progenitor cells, a plurality of mesenchymal stem cells, a plurality of endothelial progenitor cells, and a combination thereof.

In alternative embodiments, method further comprise providing a plurality of third stem cells that are different from the first cardiac stem cells and the second cardiac stem cells, and placing the plurality of third stem cells in the core region or cluster and/or the second region or the core region or cluster,

wherein optionally the plurality of third stem cell are positioned or configured: at least partially surrounding the outer surface of the second region or the core region or cluster, or at least partially around the second region or the core region or cluster,

and optionally the plurality of third stem cells or progenitor cells are positioned or configured in the core region or cluster,

and optionally the plurality of third stem cell are positioned or configured: at least partially surrounding the outer surface of the second region or the core region or cluster, or at least partially around the second region or the core region or cluster, and are also positioned or configured in the core region or cluster,

and optionally the plurality of third stem cells or progenitor cells are of non-cardiac origin, and optionally the third stem cells are mesenchymal stem cells of non-cardiac origin,

and optionally the plurality of third stem cells are selected from the group consisting of: a plurality of cardiac progenitor cells or cardiac stem cells, a plurality of mesenchymal stem cells or mesenchymal progenitor cells, a plurality of endothelial progenitor cells or endothelial stem cells, and a combination thereof.

In alternative embodiments, the third stem cells are mesenchymal stem cells of non-cardiac origin, or the third stem cells are mesenchymal stem cells of cardiac origin.

In alternative embodiments, the core region or cluster comprises cardiac progenitor cells and mesenchymal stem cells, and the second region or peripheral region and/or the plurality of third stem cells comprises endothelial stem cells.

In alternative embodiments, the methods further comprise:

(a) forming the core region or cluster by culturing the first cardiac stem cells and one or more cell types together, optionally to promote adhesion therebetween; and optionally further comprising adding the second cardiac stem cells to the core region or cluster, or to the culture of first cardiac stem cells and one or more cell types of (a), and culturing the second cardiac stem cells: to induce them to form a layer at least partially around the region or cluster, or, under conditions wherein they form a layer at least partially around the region or cluster, and

(b) the method of (a), further comprising building said cardiocluster by sequential, ordered deposition of said first and second cardiac stem cells using a tissue printer, optionally a 3-D tissue printer.

In alternative embodiments, provided are products of manufacture comprising:
a macrocellular structure, a cardiocluster of cells or an artificially configured plurality of cells as provided herein, or
a macrocellular structure, a cardiocluster of cells or an artificially configured plurality of cells made by a method as provided herein,

wherein optionally the product of manufacture comprises a drug delivery device, an implant, a catheter, a stent, or a medical device.

In alternative embodiments, provided are methods for: inducing cardiogenesis in the mammalian heart; or, tissue repair or tissue regeneration, optionally a cardiac or heart tissue repair or heart tissue regeneration, or optionally a cardiac muscle repair or tissue regeneration, a cardiac vasculature repair or tissue regeneration or a cardiac connective tissue repair or tissue regeneration, comprising:

(a) providing: a macrocellular structure, a cardiocluster of cells or an artificially configured plurality of cells as provided herein, or, a macrocellular structure, a cardiocluster of cells or an artificially configured plurality of cells made by a method as provided herein, or a product of manufacture as provided herein, and

(b) introducing into, onto or approximate to the mammalian heart, or cardiac or heart tissue, or heart muscle, or cardiac vasculature or connective tissue: the macrocellular structure, a cardiocluster of cells or an artificially configured plurality of cells of (a); or, introducing into or applying to the mammalian heart, or an individual in need thereof, the product of manufacture as provided herein,

thereby inducing cardiogenesis in the mammalian heart, or for repairing or regenerating the tissue, or the cardiac tissue, or the cardiac muscle, cardiac vasculature or cardiac connective tissue.

In alternative embodiments of the methods, the heart has an injury or dysfunction and the method is effective to treat the injury or dysfunction. In alternative embodiments, the injury or dysfunction: is an ischemic injury or a heart failure, or results from myocardial infarction (MI).

In alternative embodiments, provided are methods for treating or ameliorating a heart injury, an injury subsequent to a myocardial infarction (MI), a congenital or genetic heart defect, or a heart dysfunction, comprising:

(a) providing: a macrocellular structure, a cardiocluster of cells or an artificially configured plurality of cells as provided herein, or a macrocellular structure, a cardiocluster of cells or an artificially configured plurality of cells made by a method as provided herein, or a product of manufacture as provided herein,

(b) introducing into, onto or approximate to a mammalian heart, or administering to or applying to an individual in need thereof, the macrocellular structure, a cardiocluster of cells or an artificially configured plurality of cells of (a); or, introducing into or applying to the mammalian heart, or an individual in need thereof, the product of manufacture as provided herein,

thereby treating or ameliorating the heart injury, injury subsequent to a myocardial infarction (MI), congenital or genetic heart defect, or heart dysfunction.

In alternative embodiments, provided are uses of macrocellular structure, a cardiocluster of cells or an artificially configured plurality of cells, or a product of manufacture, selected from the group consisting of: a macrocellular structure, a cardiocluster of cells or an artificially configured plurality of cells as provided herein, a macrocellular structure, a cardiocluster of cells or an artificially configured plurality of cells made by a method as provided herein, a product of manufacture as provided herein, and a combination thereof,

for treating or ameliorating an injury subsequent to a myocardial infarction (MI), a heart injury, a congenital or a genetic heart defect, or a heart dysfunction or heart failure, or for tissue repair or tissue regeneration, or for a cardiac vasculature repair or tissue regeneration, or a cardiac connective tissue repair or tissue regeneration, or a cardiac muscle repair or tissue regeneration.

The details of one or more embodiments as provided herein are set forth in the accompanying drawings and the description below. Other features, objects, and advantages of embodiments as provided herein will be apparent from the description and drawings, and from the claims.

All publications, patents, patent applications, GenBank sequences and ATCC deposits, cited herein are hereby expressly incorporated by reference for all purposes.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The drawings set forth herein are illustrative of embodiments as provided herein and are not meant to limit the scope of the invention as encompassed by the claims.

FIG. 1 schematically illustrates an exemplary protocol for the isolation of cardiac specific CPCs, MSCs and EPCs, isolated from the human heart, to generate a macrocellular structure, and its introduction into the left ventricle for autologous cellular therapy, as further described below.

FIG. 2 schematically illustrates an exemplary protocol comprising: positive and negative c-kit fractions were collected in order to establish CPC and EPCs (c-kit⁺) separate from MSCs (c-kit⁻), as further described in Example 1, below.

FIG. 3 schematically illustrates exemplary lentiviral constructs created with use of the human phosphoglycerate kinase promoter to express enhanced green fluorescent protein (eGFP), mCherry (mCH) or mKusabira orange (mKo) proteins followed by the murine PGK promoter to express puromycin (puro) or bleocin (bleo) selectable markers, as further described in Example 1, below.

FIG. 4 illustrates an exemplary CardioCluster comprising GFP+ CPCs surrounding mCherry+ MSCs visualized by confocal microscopy; nucleic are stained with TO-PRO-3, as further described in Example 1, below.

FIG. 5 schematically illustrates an exemplary protocol for creation of macrocellular structures or artificially configured plurality of cells, the so-called “cardioclusters” as provided herein, as further described in Example 1, below.

FIG. 6 illustrates an image of an exemplary CardioCluster in midsagittal optical section; the CardioCluster comprises pmOrange+ EPCs surrounding Neptune+ MSCs and GFP+ CPC visualized by confocal microscopy; the exemplary CardioCluster is approximately 100 μm in size and composed of approximately 500 cells in total.

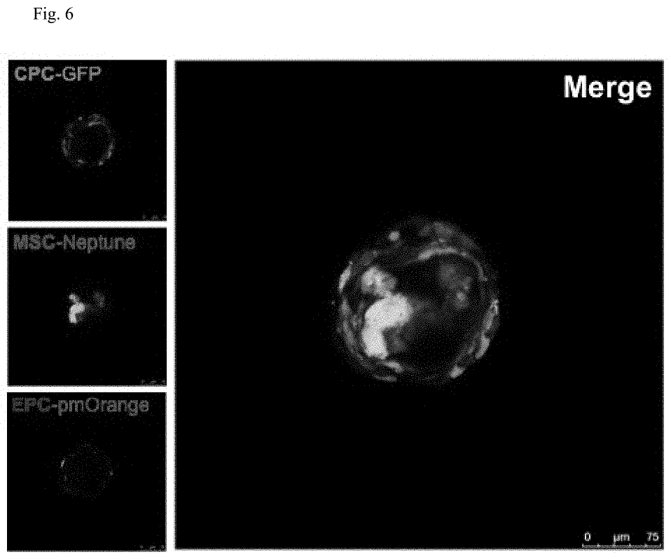

Like reference symbols in the various drawings indicate like elements.

Reference will now be made in detail to various exemplary embodiments of the invention, examples of which are illustrated in the accompanying drawings. The following detailed description is provided to give the reader a better understanding of certain details of aspects and embodiments as provided herein, and should not be interpreted as a limitation on the scope of the invention.

### DETAILED DESCRIPTION

In alternative embodiments, provided are macrocellular structures or artificially configured plurality of cells, the so-called “cardioclusters”, that can be used to treat an injured or a diseased heart. For example, in alternative embodiments, the macrocellular structures or artificially configured plurality of cells, the so-called “cardioclusters” as provided herein, provide micro-environments for enhanced stem cell proliferation and regenerative potential. The so-called “cardioclusters” as provided herein improve on the heart's limited regeneration capability, including augmenting cardiomyocyte re-entry into the cell cycle and production of new mono-nucleated myocytes, e.g., after a pathological damage such as a myocardial infarction (MI). In addition, the cardioclusters can also facilitate regeneration of cardiac vasculature or cardiac connective tissue, or both. While the invention is not limited by any particular mechanism of action, macrocellular structures as provided herein stimulate new myocyte formation that can be partially due to reserve c-kit⁺ CPCs found in complex microenvironments or niches. In vivo, CPCs retain expression of primitive cardiac transcription factors and upon activation, e.g., by macrocellular structures as provided herein, can give rise to cells of the cardiac lineages.

In alternative embodiments, the macrocellular struct as provided herein or artificially configured plurality of cells, the so-called “cardioclusters”, create the optimal stem cell environments to support communications between different cell types. In alternative embodiments, the macrocellular structures or artificially configured plurality of cells, the so-called “cardioclusters” as provided herein, can simulate the “natural” stem cell microenvironments in which communities of cells of different types exist in an organized relationship.

In alternative embodiments, the macrocellular structures or artificially configured plurality of cells, the so-called “cardioclusters” as provided herein, provide a milieu for stem cell self-renewal and differentiation, which can be tightly controlled in defined locations of all regenerative tissues, including the heart. In alternative embodiments, functions of microenvironments re-created by macrocellular structures as provided herein include the maintenance, or stimulation, of a quiescent stem cell population that are hypersensitive to stimuli such as molecular signaling and extracellular matrix (ECM) remodeling. In alternative embodiments, macrocellular structures as provided herein can create cardiac niches that can regulate symmetric or asymmetric stem cell division, as asymmetric division of CPCs creates new cardiogenic daughter cells with properties required to repopulate the damaged myocardium. In alternative embodiments, macrocellular structures as provided herein can recapitulate, or recreate, cardiac microenvironments in vivo or ex vivo, and create an enhanced cellular communication, e.g., by expression of the gap junction protein connexin 43, improving cell propagation and differentiation in vitro.

In alternative embodiments, macrocellular structures as provided herein can generate paracrine effects, for example, they can restore vasculature via endothelial precursor cells. In alternative embodiments, macrocellular structures as provided herein are rationally designed to promote efficient cardiomyogenesis.

In alternative embodiments, macrocellular structures as provided herein are formed by directed, or random, aggregation, e.g., on a matrix, e.g., an extracellular matrix (ECM), leading to variable sphere size. Macrocellular structures as provided herein can be designed to have consistent cell characterization markers, thus making the administration and treatment protocols and effects reproducible for everyday clinical practices.

In alternative embodiments, macrocellular structures as provided herein are used in cellular therapy, and for the administration or application of stem cell populations to treat cardiovascular diseases, and can promote efficient cardiac regeneration. In alternative embodiments, instead of introducing just a single cell type in an unnatural context, macrocellular structures as provided herein can restore multiple cell types simultaneously, where the cells are already organized in a manner that mimics their natural environment, and that facilitates regeneration of heart tissue that has been damaged as a consequence of cardiac disease or injury.

In alternative embodiments, macrocellular structures as provided herein can replace damaged tissue, necrotic tissue, or scar tissue in the heart with functional cardiac cell populations normally found within the myocardium. In alternative embodiments, macrocellular structures as provided herein deliver beneficial stem cells or precursor cells, such as MSCs having the ability to secrete a diverse assortment of paracrine factors.

In alternative embodiments, macrocellular structures as provided herein deliver EPCs to form microvessels, and also to support vessel maturity. EPCs and MSCs delivered by macrocellular structures as provided herein can have diverse properties and regeneration potential, and achieve long-lasting myocardial benefits that require the interaction of multiple cell types. Macrocellular structures can provide multiple stem cells types, in some embodiments, cells explanted from the human heart. Macrocellular structures as provided herein can provide CPCs that are pre-committed to the cardiovascular lineage and can produce new cardiogenic cells without inducing arrythmyogenesis, a distinct advantage over other cell types for cardiac cell therapy.

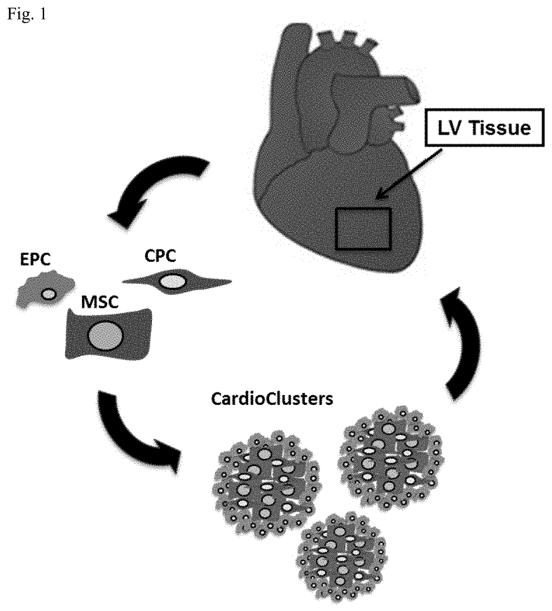

FIG. 1 schematically illustrates an exemplary protocol for the isolation of cardiac specific CPCs, MSCs and EPCs, isolated from the human heart, to generate a macrocellular structure, and its introduction into the left ventricle for autologous cellular therapy. Macrocellular structures as provided herein can integrate unique characteristics from each cell type. For example, in one embodiment, CPCs and MSCs, which prefer hypoxic conditions, form the central core of a macrocellular structure as provided herein, as illustrated in FIG. 1. In this exemplary embodiment, EPCs surround the macrocellular structure and provide for endothelial specific differentiation and production of tubular networks to reconnect with native blood vasculature, restore blood flow and aid in nutrient absorption in the heart. MSCs of the macrocellular structure can be mixed in the interior with CPCs, as MSCs secrete cell adhesion molecules such as integrins and cadherins, which are important for cellular aggregation. In this exemplary embodiment, MSCs are helpful in supporting EPC maturation and functionality by secretion of paracrine factors to promote long-term EPC-dependent vasculogenesis. In alternative embodiments, macrocellular structures can form a microcosm that will support cell survival and proliferation, making their administration an ideal route for cardiac restoration.

In alternative embodiments, macrocellular structures provide the regenerative potential of stem cells in a clinical setting, e.g., providing stem cells capable of performing a variety of mechanisms for myocardial repair. In alternative embodiments, different exemplary macrocellular structures as provided herein are rationally designed and based on known characteristics and functions of CPCs, MSCs and EPCs. Macrocellular structures described herein can deliver to a patient cardiogenic stem cells for treating a diseased heart. Macrocellular structures as provided herein can recapitulate the complex network found within stem cell microenvironments.

### Methods for Administering CardioClusters

In alternative embodiments, macrocellular structures or artificially configured plurality of cells as provided herein, the so-called “cardioclusters”, are administered to induce cardiogenesis in a mammalian (e.g., a human) heart, or for treating or ameliorating a heart injury, a congenital or genetic heart defect, or a heart dysfunction. The so-called “cardioclusters” as provided herein can be administered by any means known in the art, for example, by local injection (including e.g., intracoronary, intramyocardial and endocardial routes), infusion or equivalent techniques. In alternative embodiments, so-called “cardioclusters” as provided herein are administered, or delivered in vivo, through coronary arteries, coronary veins, or peripheral veins; or, alternatively, via direct intramyocardial injection using a surgical, transendocardial, or transvenous approach; see e.g., Rosen et al., J Am Coll Cardiol. 2014; 64(9):922-937; Perin et al. Nat Clin Pract Cardiovasc Med. 2006 March; 3 Suppl 1:S110-3. In one embodiment, catheters are used to administer, or deliver, the so-called “cardioclusters” as provided herein, e.g., ND INFUSION CATHETER™ Translational Research Institute, aka TRI Medical (Frankfurt, Germany).

Techniques for delivering nucleic acids (e.g., gene therapy) to the heart can also be adapted for administration of so-called “cardioclusters” as provided herein. Examples and descriptions of such protocols and techniques that can be used, and adapted, to practice compositions and methods as provided herein in vivo are e.g., Rasmussen (2011) Circulation, vol 123, pgs 1771-1779; Bridges et al., Annals of Thoracic Surgery, 73: 1939-1946 (2002); Wang, et al., Catheter, Circulation, 2009, pp. S238-S246, vol. 120, suppl. 1. Vulliet, et al., Lancet, Mar. 6, 2004; WO 2005/030292 (Apr. 7, 2005); WO 2005/027995; U.S. Patent application publication 20060258980; U.S. Pat. Nos. 7,722,596; 8,158,119; 8,846,099.

Additionally, materials or delivery adjuvants can be used to enhance cell retention and their longevity once delivered to a heart, e.g., by administration with or formulated with (e.g., mixed with) a gel or a hydrogel, such as a chitosan-based hydrogel, e.g., as described in Kurdi et al. Congest Heart Fail. 2010 May-June; 16(3):132-5, or any biocompatible scaffold, e.g., as described in U.S. Pat. Nos. 8,871,237; 8,753,391; 8,802,081; 8,691,543, or Pagliari et al. Curr Med Chem. 2013; 20(28):3429-47, or biomimetic support , e.g., as described in Karam et al. Biomaterials. 2012 August; 33(23):5683-95.

### 3-D Printing and Three-Dimensional Living Biological Tissue

Provided are three-dimensional living biological tissues made using the so-called “cardioclusters” as provided herein. In one embodiment, the cardioclusters are used in a “bio-printing” process to generate a spatially-controlled cell pattern using a 3D printing technology. Any bio-printing or bio-fabricating process known in the art can be used, e.g., as described in U.S. Pat. App. Pub. Nos. 20140099709, 20140093932, 20140274802, 20140012407, 20130345794, 20130190210 and 20130164339; and U.S. Pat. No. 8,691,974.

For example, in one embodiment, a printer cartridge is filled with a suspension of “cardioclusters” as provided herein and a “smart gel”; the, alternating patterns of the smart gel and cells, or cardioclusters, are printed using a standard print nozzle. In alternative embodiment, a NovoGen (San Diego, Calif.) MMX™, or Organovo Holdings, Inc., bioprinters are used for 3D bioprinting. This and equivalent “bio-printers” can be optimized to “print”, or fabricate, skin tissue, heart tissue, and blood vessels, and other basic tissues, all of which are suitable for surgical therapy and transplantation.

### Kits

Provided are kits comprising compositions as provided herein and methods as provided herein, including so-called “cardioclusters” as provided herein, or any combination thereof. As such, kits, cells, instructions and the like are provided herein.

The invention will be further described with reference to the following examples; however, it is to be understood that the invention is not limited to such examples.

### EXAMPLE 1: MAKING AND USING “CARDIOCLUSTERS”

The following example describes making and using the so-called “CardioClusters” as provided herein, for e.g., enhancing myocardial repair with the so-called “CardioClusters”. In summary: provided are macrocellular structures or artificially configured plurality of cells, the so-called “cardioclusters” as provided herein, which in alternative embodiments are three-dimensional (3-D) micro-environments comprising at least three, or more, defined stem cell populations, e.g., from the human heart, including c-kit+ cardiac progenitor cells (CPCs), CD90+/CD105+ mesenchymal stem cells (MSCs) and CD133+ endothelial progenitor cells (EPCs).

The size of the macrocellular structures, the artificially configured plurality of cells, or the so-called “cardioclusters” as provided herein can be controlled by the quantity of cells used to create the cluster, allowing them to be infused into the heart without being reduced to single cell suspensions (as can be the case for cardiosphere-derived cells, where the structural and cell-cell contact information is lost when delivered).

In alternative embodiments, the cardiocluster cells are combined into a rationally designed cluster with MSCs and CPCs in the central core and EPC forming the outer layer. In these embodiments, the EPCs play a vital role in forming a neovasculature that can connect the cardiocluster cells to living heart tissue not damaged by ischemia and allow for revascularization of the damaged myocardium.

We have found that EPCs have increased resistance to apoptotic stress and therefore are an ideal cell type for the exterior of exemplary macrocellular structures or artificially configured plurality of cells, the so-called “cardioclusters” as provided herein. In vitro we have shown that EPCs are better able to form tubular networks on matrigel-coated plates compared to either CPCs or MSCs. MSCs reinforce the 3-D structure by releasing growth factors that attract and maintain cells within the cluster. Clinically, macrocellular structures or artificially configured plurality of cells, the so-called “cardioclusters”, can broaden the application of cell types into a single structure to increase engraftment, mitigate inflammation and prevent the progression of heart failure.

In alternative embodiments, macrocellular structures as provided herein exhibit enhanced proliferation, survival and cardiac commitment relative to cardiospheres or single cell populations. In alternative embodiments, provided are adaptable protocols making macrocellular structures as provided herein using single and combinatorial stem cells. In alternative embodiments, provided are macrocellular structures capable of creating a microenvironment of stem cells to promote cell survival and proliferation, e.g., through paracrine dependent mechanisms and direct differentiation of stem cells for efficient cardiac repair.

### Methodological Details:

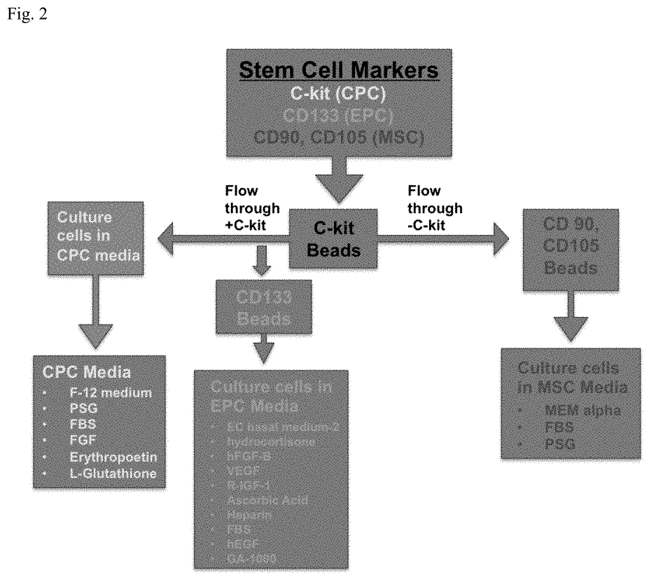

Isolation of human stem cells: Stem cells were isolated from both whole fetal hearts and adult tissue samples from patients undergoing surgery for left ventricular assist device, or LVAD (a mechanical pump). Tissue was digested in collagenase and incubated with c-kit labeled beads and subjected to magnetic activated cell sorting. The positive and negative c-kit fractions were collected in order to establish CPC and EPCs (c-kit⁺) separate from MSCs (c-kit⁻) as schematically illustrated in FIG. 2. Cells types were validated by flow cytometric analysis to express markers that define them as CPCs, EPCs and MSCs after sorting and expansion.

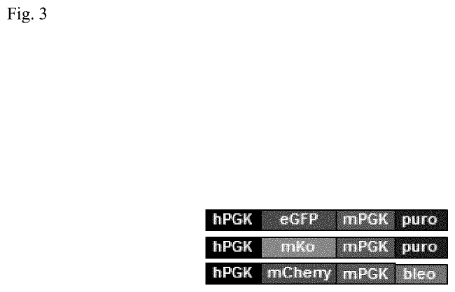

Lentiviral based labeling of stem cells for in vitro and in vivo use. Lentiviral constructs were created with use of the human phosphoglycerate kinase promoter to express enhanced green fluorescent protein (eGFP), mCherry (mCH) or mKusabira orange (mKo) proteins followed by the murine PGK promoter to express puromycin (puro) or bleocin (bleo) selectable markers as illustrated in FIG. 3. In alternative embodiment, other detectable agents or proteins are used, e.g., including Green Flourescent Protein (GFP), Neptune, mCherry, pmOrange, Cerulean or Plum.

All stem cells are transduced with lentiviruses prior to experimental protocols, and express fluorescent markers within 48 hours. If expression of fluorophore is less than 80% after flow cytometric analysis cells can be purified for fluorescent tags by treatment with either puro or bleo. These fluorescent markers were chosen due to non-overlapping spectral emissions for flow activated cell sorting methods. Specific antibodies to detect eGFP, mCherry and mKO proteins in biochemical assays such as immunoblotting, immunocytochemistry and immunohistochemistry can be used for identification. In alternative embodiment, other detectable agents or proteins are used, including antibodies that bind to them, e.g., including Green Flourescent Protein (GFP), Neptune, mCherry, pmOrange, Cerulean or Plum.

Creation of CardioClusters. In alternative embodiments, three human cell types (CPCs, MSCs, and EPCs) from both fetal and adult samples were used to create the macrocellular structures or artificially configured plurality of cells, the so-called “cardioclusters” as provided herein, with attributes that are beneficial for tissue repair.

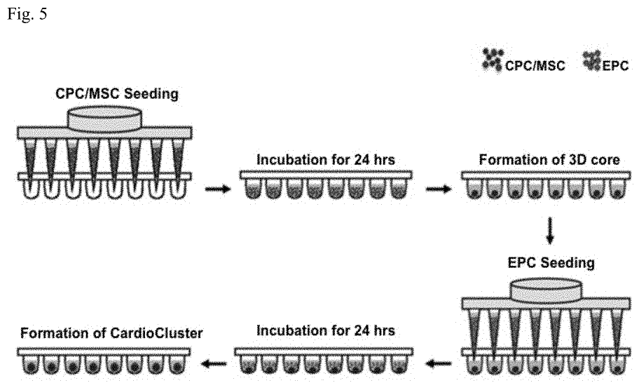

In exemplary methods for making macrocellular structures as provided herein, “ultra-low adherent” 96-well plates, or equivalents, are used, or alternatively, a methylcellulose hydrogel is placed at the bottom of a 96-well plate (or equivalents) as schematically illustrated in FIG. 5. FIG. 5 schematically illustrates an exemplary protocol for creation of macrocellular structures or artificially configured plurality of cells, the so-called “cardioclusters” as provided herein. Cell aggregation was promoted by seeding of CPCs/MSCs on methylcellulose (MC), a hydrophilic hydrogel that inhibits cell attachment. EPCs were added 24 hours later to create the outer layer of the CardioCluster. Adapted from (Lee, W-Y, 2011; Biomaterials).

In alternative embodiment, CPCs and MSCs are seeded for 24 hours, or for between 1 to 3 days, or for between 1 to 7 days, for formation of the core. EPCs are seeded for an additional 24 hours, or for between 1 to 3 days, or for between 1 to 7 days, to form the outer layer, of the core. In one embodiment, after a total of 48 hours macrocellular structures are collected. Macrocellular structures can be dissociated e.g., by incubation with collagenase or equivalents to achieve single cell suspensions of the three cell types and individual stem populations can be sorted based on expression of GFP, mCH and mKo to analyze cells in and out of the microenvironment.

Stem cell proliferation and growth is enhanced by paracrine secretion of growth factors and extracellular matrix proteins in CardioClusters. Measurement of cell surface area, relative length to width ratios and roundness of cells were used to characterize the three distinct stem cell populations. In vitro assessments included metabolic activity (MTT based assay), cell proliferation (CyQuant assay), proportion of cells undergoing DNA synthesis (BrdU incorporation), and cell cycle analysis (propidium iodide staining and flow cytometry analysis). Antibody detection followed by immunofluorescence was used to detect ECM proteins such as collagen type I, collagen type III, laminin, fibronectin, and cadherin proteins using a confocal laser scanning microscope (CLSM). Experiments were performed on fetal and adult CardioClusters, collagenase digested CardioClusters, cardiospheres, 2D co-culture of the three populations, as well as single cell populations. All experiments can be performed in triplicate with statistical analyses to establish clear significance of biological parameters where applicable.

Apoptosis and cell death is reduced by incorporation of cells into CardioClusters. Stem cell survival were analyzed by trypan blue exclusion assay after successive days of plating to determine live versus dead cells during basal conditions and after apoptotic challenge. As core cells within the CardioCluster may have reduced access to nutrients and oxygen, confocal z-stack images were acquired using a live/dead fluorescent-based assay. Treatment with pro-apoptotic stimuli was performed with hydrogen peroxide, staurosporine, hypoxia and serum starvation. Functional readouts of apoptosis and cell death can be determined by labeling cells with Annexin V and a nuclear dye (sytox blue, TO-PRO-3, propidium iodide) to determine early and late stages of apoptosis by flow cytometric analysis. In parallel, CardioClusters as provided herein, cardiospheres, dissociated stem cells and control stem cell populations were co-cultured with neonatal rat cardiomyocytes (NRCM) in low serum conditions in the ratio of 1:40 for 7 days to simulate a stressed environment and determine the protective effect of secreted paracrine factors from stem cells. Negatively sorting out NRCMs by flow cytometric analysis and staining for Annexin V and nuclear dye were used to quantitate cardiomyocyte cell death after incubation with stem cells. Furthermore, NRCMs were stained with Terminal deoxynucleotidyl transferase dUTP nick end labeling (TUNEL) to label apoptotic/dying cells after co-culture.

Cardiac lineage commitment, angiogenesis and paracrine secretion is increased after differentiation stimuli as well as co-culture of CardioClusters with cardiomyocytes. Differentiation was induced by dexamethasone treatment for 7 days and cells were characterized for expression of differentiation markers to validate lineage commitment including cardiac transcription factors: GATA-4, myocyte enhancer factor-2 (Mef-2) and Nkx2.5; smooth muscle markers: GATA-6, myosin heavy chain 11 (Myh11), α-smooth muscle actin (α-SMA); mature endothelial markers: von Willebrand Factor (vWF), platelet endothelial cell adhesion molecule (PECAM-1 or CD31) and vascular endothelial cadherin (VE-Cadherin) by quantitative real time polymerase chain reaction (qRT-PCR). Antibody staining followed by immunofluorescence validated protein expression and confocal microscopy as previously published in mouse and human derived CPCs.

In parallel, cells were co-cultured with neonatal rat cardiomyocytes (NRCMs) in the ratio of 1:40 for 7 days to promote cardiac specific differentiation. The ability of stem cells to form tubular networks and structures was analyzed after plating on Matrigel-coated wells (25,000 cells/well). Expression of stem cell markers before and after differentiation stimuli confirmed if stem cells retain naïve cell characteristics relative to more committed markers. Unique paracrine profiles of CardioClusters relative to cardiospheres and individual cell lines was determined from human cytokine and inflammation PCR arrays. Differential up-regulation of paracrine factors known to signal pro-survival pathways while reducing inflammation was considered, such as anti-inflammatory factors that inhibit adverse T-cell responses that are up regulated in the acutely infarcted heart.

After candidate genes are determined, confirmatory qRT-PCR can be performed. Enzyme-Linked Immunosorbent Assays (ELISA) was used to validate secretion of proteins in the supernatant from CardioCluster, as well as control cells. NRCM survival was compared with paracrine secretion profiles after differentiation stimuli by flow cytometric analysis for apoptosis and cell death labeling.

In alternative embodiments, administration of CardioClusters as provided herein enhanced stem cell survival and expansion via the physical interactions between cell types and the suggested paracrine effect from EPCs and MSCs. Upon differentiation, cells within the CardioClusters as provided herein displayed enhanced matrigel tubular formation from EPCs, secretion of paracrine factors, and differentiation capabilities compared to single stem cell populations, which had a positive impact on survival of stem cells and cardiomyocytes in a co-culture manner.

Co-culture approach with NRCMs was used to confirm the ability of stem cells to differentiate into cardiomyocytes relative to smooth muscle and endothelial cells by using human specific qRT-PCR primers and antibodies to distinguish from rat derived cardiomyocytes. In alternative embodiment, the order of layering cells in the CardioClusters as provided herein was interchangeable based on the ability of EPCs to survive and secrete paracrine factors in hypoxic conditions, a characteristic of most stem cell populations. Therefore, determining the cell that is most resistant to cell death, such as in the presence of hydrogen peroxide, may be an alternative approach to determine the cell type used to form the outer core of any particular CardioCluster.

The ratio of cells to be constructed can dictate the size of the cluster and the level of hypoxia in the center of the core. The size of the CardioCluster as provided herein can be controlled, e.g., based on cell number; this can determine the optimal size of a CardioCluster for not only survival and growth of stem cells, but for the impact of intramyocardial injection, and it efficacy for any particular purpose.

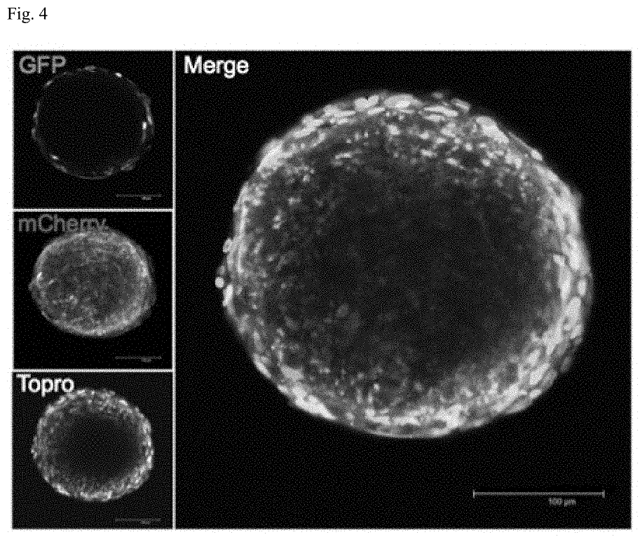

Fixation of CardioClusters for confocal microscopy reduces intensity of expressed fluorophores. Exemplary protocols include aspects of standard protocols that require fixation and permeabilization of the cell membrane to allow for nuclear staining, such as is required when staining with TO-PRO and SYTOX. Alternatively, a live cell nuclear stain such as DRAQ5 can be used to label non-fixed cells. DRAQ5 is a membrane-permeable dye with a high affinity for double-stranded DNA. FIG. 4 illustrates images of a fixed CardioCluster of the invention in midsagittal optical section; the CardioCluster comprises GFP+ CPCs surrounding mCherry+ MSCs visualized by confocal microscopy. Nuclei were stained with TO-PRO-3. To compensate for oversaturation of nuclei on the surface of the CardioCluster the image represents a balance between very bright and dimly stained nuclei. This exemplary CardioCluster is approximately 200 μm in size and composed of approximately 10,000 cells in total.

In alternative embodiments, CardioCluster as provided herein restore myocardial structure and function after intramyocardial injection, and in some situations, can do so better than cardiospheres or single/multiple cell suspensions. Traditional stem cell therapy for treatment of heart damage is hindered by poor survival of delivered stem cells and inefficient commitment and engraftment. Acute MI is characterized by adverse inflammation and secretion of detrimental growth factors that promote fibrotic scar formation. In effect, irreversible myocardial damage decreases hemodynamic function and leads to heart failure. In response to MI, the myocardium has adapted modest endogenous regenerative mechanisms to replace lost or damaged cells. CardioCluster as provided herein provide therapeutic advantages in delivering stem cells relative to cardiospheres, and single or combined cell populations. CardioClusters transplanted in vivo demonstrate the combinatorial roles of CPCs, MSCs and EPCs to enhance cardiogenic repair by improvement of commitment and secretion of protective factors.

Methodological details: CardioClusters as provided herein were injected in the myocardial border zone of an acutely infarcted immunodeficient NOD-SCID mouse heart by ligation of the Left Anterior Descending (LAD) artery prior to delivery and using phosphate buffered saline (PBS) as a vehicle. Negative control groups were maintained by injection of phosphate buffered saline alone (no cellular treatment). Positive controls were maintained by performing sham operations (opening and closing of the chest). PBS and cell treated groups were evaluated for comparable infarct size and impaired ejection fraction (EF) three days post-surgery relative to sham controls using echocardiography. Mice with less than 50% of the left ventricle infarcted within three days of surgery were excluded from the experiment. Additionally, mice were sacrificed from each group to determine the efficiency of injection of CardioClusters as provided herein. Control groups for CardioClusters included cellular treatment with CPCs, MSCs, and EPCs that were not pre-formed in clusters but maintained in co-culture or single populations for the same duration as the experimental group, as well as cardiospheres. Injection of control groups for CardioCluster delivery included combinatorial therapy of non-fused CPCs and MSCs or EPCs and MSCs.

Survival, engraftment, and persistence are improved by incorporation of stem cells into CardioClusters: After intramyocardial injection of CardioClusters as provided herein, detection and quantitation of stem cell treatments was determined by immunohistochemistry by labeling CardioClusters that express single fluorescent proteins or co-expression of both GFP and mCherry in stem cell chimeras to determine engraftment relative to control groups. Morphological analysis of stem cells within the myocardium was analyzed to determine if the formation of CardioClusters is retained in spherical formation or dissociated into single cell types.

Cardiac structure and function is enhanced by injection of CardioClusters. Cell treatment to affect cardiac function was measured each week after infarction and treatment up to four weeks to validate the short-term effects of CardioClusters as provided herein. Prior studies have recognized that CPC based cell therapy showed significant increases in EF and fractional shortening as early as the four week time point. In our study, if significant differences are observed at early time points after surgery, mice were subjected to in vivo hemodynamics by inserting a pressure-volume catheter through the carotid artery to enter the left ventricle. Measurements obtained determined end diastolic and end systolic pressure as well as developed pressure (maximum and minimum mmHg/seconds) to evaluate cardiac functional parameters using a VEVO 2200™ echocardiographic machine. Remaining mice in each group were maintained to evaluate the long-term effect of CardioClusters, such as every two weeks following hemodynamics until twelve weeks after injection or longer. Before completion of the experiment, all mice can be subjected to in vivo hemodynamics, and sacrificed by retroperfusion to fix the heart in diastole with formalin before embedded in paraffin to create sections for immunohistochemistry. Tibia length and heart, body and lung weights can be measured to determine relative physiological health such as congestive heart failure (Lung weight/body weight) and cardiac hypertrophy (heart weight/body weight or tibia length). For analysis of longitudinal assessment, animal heart function can be statistically evaluated using two-way ANOVA to determine differences in cardiac function over time.

Cardiac improvement is correlated with inhibition of adverse remodeling after infarction such as reduced scar formation and prevention of dilation. Similar to cardiac functional analysis, dilation of the left ventricle can be determined by echocardiography, which will accurately quantify ventricular volume and mass. Tissue Doppler imaging was used to test the applicability of our cellular treatments to affect myocardial tissue strain and wall stress, by analyzing the motion of diverse structural components of the heart. Anterior wall thickness was measured to determine the rescue of infarcted myocardium by injection of CardioClusters as provided herein in the anterior wall of the myocardium. Formation of fibrotic scarring and left ventricular infarct size was analyzed by Masson's Trichome staining and immunohistochemistry to quantify the percentage of surviving myocardium relative to scar formation after cellular treatment using antibodies against tropomyosin (cardiomyocytes) and pro-collagen and collagen I and III (scar tissue). Fibrosis and infarct size measurements at an early and late time point was analyzed with at least five mice per group and one-way ANOVA statistical analysis to determine significance between experimental groups.

Direct commitment of CardioClusters is improved as evidenced by increased formation of myocytes, endothelial and vascular smooth muscle cells and structures. Cells in CardioClusters were further analyzed for morphology and expression of cardiac progenitor transcription factors GATA-4, Mef-2 and Nkx2.5 and c-kit, MSC markers CD105 and CD90 and EPC expressing CD133 to determine if cells remain stem cell characteristics after different time points after injection. To confirm direct commitment, co-labeling with proteins that define sarcomeres and cardiomyocyte structure was performed with labeling of a-sarcomeric actin and/or tropomyosin with fluorescent transgenes. Furthermore, identification of stem cell derived supporting cells of the cardiogenic lineage, GFP, mKO and mCH cells were co-stained with markers of mature vascular smooth muscle and vascular endothelium identified with antibodies against α-SMA, GATA-6, smooth muscle 22 and vWF. Fluorescently tagged cells that are also co-labeled for mature cardiac markers were analyzed for telomere length to define young cardiac cells relative to the surviving mature myocytes using a telomere FISH protocol on immunohistochemical sections routinely performed in our laboratory and in this published report.

Endogenous stem cell population proliferation, survival and recruitment are enhanced by injection of CardioClusters. Injection or administration of CardioClusters as provided herein resulted in mobilization of endogenous stem cells, including delivery of growth factors such as leukocyte inhibitory factor and stromal derived growth factor, thus promoting beneficial effects on cardiac function. Recruitment of stem cells in the infarcted myocardium following CardioCluster administration or treatment were identified by immunohistochemistry in order to quantitate the number of c-kit⁺ cells that are not expressing fluorescent markers within the infarcted region.

In alternative embodiments, it can be important to determine the numbers of c-kit⁺ cells that co-express hematopoietic markers such as CD45 or CD34 (myeloid progenitor marker) to distinguish between cardiac resident c-kit⁺ cells or cells mobilized from the bone marrow. Assessment of proliferation and expansion of c-kit⁺ stem cells was evaluated for proliferation markers such as Ki67, proliferating cell nuclear antigen (PCNA) and increased cells undergoing mitosis by detection of phosphorylated histone H3 in both the fluorescent negative and fluorescent positive populations within the heart. These studies were performed at the early time point (one to four weeks after MI) to determine if CardioClusters supply a paracrine effect relative to delivery of single or combinatorial stem cell populations.

Injection or administration of CardioClusters as provided herein resulted in rescuing or salvaging existing cardiomyocytes; this was evaluated by using a terminal deoxynucleotidyl transferase dUTP nick end labeling (TUNEL) assay in tropomyosin/a-sarcomeric actin positive cardiomyocytes at an early time point after infarction and intramyocardial injection, for example, at 3 to 7 days after MI. Delivery of chimeric cells as provided herein promoted endogenous regeneration in addition to improving cardiac repair via enhanced lineage commitment. Based on images of two-cell CardioClusters, the sizes of cells and structures are increased, which will substantially improve the retention of our cellular treatments.

For the injection of CardioClusters as provided herein, the correlation of cell number (no greater than 10,000 cells total) and size of the sphere was evaluated to determine the optimal size of the sphere for any particular indication (e.g., no greater than 200 micrometers to fit through a 27 gauge needle as previously reported with cardiospheres and core-shell bodies) to promote regeneration of the heart.

Although the invention has been described in the context of certain embodiments, it is intended that the patent will not be limited to those embodiments; rather, the scope of this patent shall encompass the full lawful scope of the appended claims, and lawful equivalents thereof.

1-14. (canceled)

15. A method for:
inducing cardiogenesis in a mammalian heart in an individual in need thereof,
inducing cardiac or heart tissue repair or heart tissue regeneration in an individual in need thereof,
treating or ameliorating a heart injury in an individual in need thereof,
treating or ameliorating a congenital or genetic heart defect in an individual in need thereof,
comprising:
(a) providing
a cardiocluster of cells comprising:
(a) a core region consisting of cardiac progenitor cells and mesenchymal stem cells (MSCs); and
(b) a peripheral region positioned at least partially surrounding the core region, the peripheral region comprising a plurality of CD133+ endothelial progenitor cells (EPCs),
wherein the cardiac progenitor cells and mesenchymal stem cells (MSCs) are substantially located or positioned in the core region, and the peripheral region substantially comprises the CD133+ endothelial progenitor cells; and
(b) introducing into, onto or approximate to the mammalian heart, or the cardiac or heart tissue, of the individual in need thereof the the cardiocluster of cells of (a),
thereby inducing cardiogenesis in the mammalian heart, or inducing cardiac or heart tissue repair or heart tissue regeneration in the individual in need thereof, or treating or ameliorating the heart injury or the congenital or genetic heart defect in the individual in need thereof.

16. The method of claim 15, wherein heart has an injury or dysfunction and the method is effective to treat the injury or dysfunction.

17. The method of claim 16, wherein injury or dysfunction: is an ischemic injury or a heart failure, or results from myocardial infarction (MI), or the heart injury comprises an injury subsequent to a myocardial infarction (MI).

18-19. (canceled)

20. The method of claim 15, wherein the heart tissue repair comprises a cardiac vasculature repair or cardiac vasculature regeneration,

21. The method of claim 15, wherein the heart tissue repair comprises or a cardiac connective tissue repair or tissue regeneration.

22. The method of claim 15, wherein the cardiac progenitor cells or the CD133+ endothelial progenitor cells or mesenchymal stem cells (MSCs) are of human origin.

23. the method of claim 15, wherein the cardiac progenitor cells, the CD133+ endothelial progenitor cells and mesenchymal stem cells are of human origin.

24. The method of claim 15, wherein the cardiac progenitor cells of the core region comprise c-kit+ cardiac progenitor cells (CPCs).

25. The method of claim 15, wherein the peripheral region substantially comprises CD133+ endothelial progenitor cells (EPCs).

26. The method of claim 15, wherein the cardiocluster of cells are made by a method comprising:
(a) providing a plurality of cardiac progenitor cells and mesenchymal stem cells (MSCs),
(b) providing a plurality of CD133+ endothelial progenitor cells,
(c) forming or fabricating a core region of cells consisting of the plurality of mesenchymal stem cells (MSCs) and cardiac progenitor cells; and
(d) (i) forming or fabricating a peripheral region comprising: a layer comprising the plurality of the CD133+ endothelial progenitor cells at least partially around the core region of cells, or
(ii) forming or fabricating a peripheral region by placing the CD133+ endothelial progenitor cells at least partially around the outer surface of the core region of cells,
thereby forming the cardiocluster of cells, the macrocellular structure or the artificially configured plurality of cells.

27. A method for:
inducing cardiogenesis in a mammalian heart in an individual in need thereof,
inducing cardiac or heart tissue repair or heart tissue regeneration in an individual in need thereof,
treating or ameliorating a heart injury in an individual in need thereof,
treating or ameliorating a congenital or genetic heart defect in an individual in need thereof,
comprising:
introducing into, onto or approximate to the mammalian heart, or the cardiac or heart tissue, of the individual in need thereof a cardiocluster of cells of (a),
wherein the cardiocluster of cells comprises:
(a) a core region consisting of cardiac progenitor cells and mesenchymal stem cells (MSCs); and
(b) a peripheral region positioned at least partially surrounding the core region, the peripheral region comprising a plurality of CD133+ endothelial progenitor cells (EPCs),
wherein the cardiac progenitor cells and mesenchymal stem cells (MSCs) are substantially located or positioned in the core region, and the peripheral region substantially comprises the CD133+ endothelial progenitor cells,
wherein the cardiocluster of cells are made by a method comprising:
(i) providing a plurality of cardiac progenitor cells and mesenchymal stem cells (MSCs),
(ii) providing a plurality of CD133+ endothelial progenitor cells,
(iii) forming or fabricating a core region of cells consisting of the plurality of mesenchymal stem cells (MSCs) and cardiac progenitor cells; and
(iv) (1) forming or fabricating a peripheral region comprising: a layer comprising the plurality of the CD133+ endothelial progenitor cells at least partially around the core region of cells, or
(2) forming or fabricating a peripheral region by placing the CD133+ endothelial progenitor cells at least partially around the outer surface of the core region of cells,
thereby forming the cardiocluster of cells, the macrocellular structure or the artificially configured plurality of cells,
thereby inducing cardiogenesis in the mammalian heart, or inducing cardiac or heart tissue regeneration in the individual in need thereof, or treating or ameliorating the heart injury or the congenital or genetic heart defect in the individual in need thereof.

28. The method of claim 27, wherein the heart tissue repair comprises a cardiac vasculature repair or cardiac vasculature regeneration,

29. The method of claim 27, wherein the heart tissue repair comprises or a cardiac connective tissue repair or tissue regeneration.

30. The method of claim 27, wherein the cardiac progenitor cells or the CD133+ endothelial progenitor cells or mesenchymal stem cells (MSCs) are of human origin.

31. the method of claim 27, wherein the cardiac progenitor cells, the CD133+ endothelial progenitor cells and mesenchymal stem cells are of human origin.

32. The method of claim 27, wherein the cardiac progenitor cells of the core region comprise c-kit+ cardiac progenitor cells (CPCs).

33. The method of claim 27, wherein the peripheral region substantially comprises CD133+ endothelial progenitor cells (EPCs).
